(19) United States
(12) Patent Application Publication
(10) Pub. No.: US 2026/0024627 A1
(43) Pub. Date: Jan. 22, 2026

(54) NON-CONTRASTIVE AUXILIARY LOSS BASED LEARNING FOR MACHINE LEARNING ENABLED MOLECULAR ANALYSIS

(71) Applicant: Genentech, Inc., South San Francisco, CA (US)

(72) Inventors: Jae Hyeon LEE, Boston, MA (US); Yao-Yu LIN, Brooklyn, NY (US); Michael R. Maser, South San Francisco, CA (US); Ji Won PARK, Foster City, CA (US); Andrew Martin WATKINS, San Francisco, CA (US)

(21) Appl. No.: 19/287,688

(22) Filed: Jul. 31, 2025

Related U.S. Application Data
(63) Continuation of application No. PCT/US2024/013525, filed on Jan. 30, 2024.
(60) Provisional application No. 63/482,550, filed on Jan. 31, 2023.

(51) Int. Cl. G16C 20/70; G16C 20/30

(52) U.S. Cl. CPC G16C 20/70; G16C 20/30

(57) ABSTRACT

A molecular analysis model may be trained to generalize across multiple molecular geometries by modifying a three-dimensional structure of one or more conformers of a molecule to generate. for each conformer. a plurality of augmented samples. The molecular analysis model may be trained to generate an embedding for each augmented sample while minimizing a difference between the plurality of embeddings resulting therefrom. Furthermore. the molecular analysis model may be trained to determine, based at least on the plurality of embeddings. a value of a molecular property for the molecule. The trained molecular analysis model may be applied in the determination of the value of the molecular property for another molecule.

### CROSS REFERENCE TO RELATED APPLICATION

This application claims priority to U.S. Provisional Application 63/482,550, entitled “NON-CONTRASTIVE AUXILIARY LOSS BASED LEARNING FOR MACHINE LEARNING ENABLED MOLECULAR ANALYSIS” and filed on Jan. 31, 2023, the disclosure of which is incorporated herein by reference in its entirety.

### TECHNICAL FIELD

The subject matter described herein relates generally to molecular analysis and more specifically to machine learning enabled techniques for molecular analysis.

### INTRODUCTION

A molecule is a group of two more atoms held together by chemical bonds. Molecules form the smallest identifiable unit into which a pure substance can be divided while still retaining the composition and chemical properties of that substance. One example of a molecule is a small molecule, which is a low-weight compound having a molecular weight between approximately 100 Daltons and 1000 Daltons. Small molecule therapeutics, which modulate biochemical processes to diagnose, treat, and prevent a gamut of illnesses, have been a cornerstone in modern pharmacology due to a number of compelling advantages. For example, small molecule drugs are capable of penetrating cell membranes to reach intracellular targets. Moreover, small molecule drugs are adaptable to a wide variety of therapeutic applications. For instance, a small molecule drug may be formulated as pills and capsules, intravenous or subcutaneous injectables, inhalational medicines, or suppositories. The development of the small molecule drug may further extend to tailoring various pharmacokinetic properties including liberation, absorption, distribution, metabolism, potency, efficacy, phenotypic effects, and excretion.

By contrast, large molecules (also known as biopharmaceuticals, biologicals, or biologics) can range between approximately 3000 Daltons and 150,000 Daltons in molecular weight. Large molecule drugs are often derivatives of natural human proteins, which modulate many essential cellular functions such as enzymatic reactions, transport of molecules, regulation and execution of a number of biological pathways, cell growth, proliferation, nutrient uptake, morphology, motility, intercellular communication, and/or the like. It is common for a single large molecule to have more than 1,300 amino acid residues, which are linked by peptide bonds to form one or more polypeptide. Due to their size and complexity, large molecule drugs are recombinantly produced by engineered cells instead of being chemically synthesized like the majority of small molecule drugs. Moreover, large molecule therapeutics are usually delivered through injection or infusion due to the ineffectiveness of oral administration. The development of a large molecule drug may entail designing one or more sequences of amino acid residues capable of binding to a target (e.g., a protein, a nucleic acid, and/or the like) with sufficient specificity and absent undesirable traits such as immunogenicity, self-association, instability, and/or the like.

### SUMMARY

Systems, methods, and articles of manufacture, including computer program products, are provided for molecular machine learning (MolML) tasks with non-contrastive auxiliary task learning. In one aspect, there is provided a system for machine learning enabled molecular property analysis. The system may include at least one processor and at least one memory. The at least one memory may include program code that provides operations when executed by the at least one processor. The operations may include: generating, for a first conformer of a first molecule, a first plurality of augmented samples by at least modifying a first three-dimensional structure of the first conformer; training a molecular analysis model to generate an embedding for each augmented sample in the first plurality of augmented samples while minimizing a difference between a first plurality of embeddings resulting therefrom, and determine, based at least on the first plurality of embeddings, a value of a molecular property for the first molecule; and applying the trained molecular analysis model to determine the value of the molecular property for a second molecule.

In another aspect, there is provided a method for machine learning enabled molecular property analysis. The method may include: generating, for a first conformer of a first molecule, a first plurality of augmented samples by at least modifying a first three-dimensional structure of the first conformer; training a molecular analysis model to generate an embedding for each augmented sample in the first plurality of augmented samples while minimizing a difference between a first plurality of embeddings resulting therefrom, and determine, based at least on the first plurality of embeddings, a value of a molecular property for the first molecule; and applying the trained molecular analysis model to determine the value of the molecular property for a second molecule.

In another aspect, there is provided a computer program product for machine learning enabled molecular property analysis. The computer program product may include a non-transitory computer readable medium storing instructions that cause operations when executed by at least one data processor. The operations may include: generating, for a first conformer of a first molecule, a first plurality of augmented samples by at least modifying a first three-dimensional structure of the first conformer; training a molecular analysis model to generate an embedding for each augmented sample in the first plurality of augmented samples while minimizing a difference between a first plurality of embeddings resulting therefrom, and determine, based at least on the first plurality of embeddings, a value of a molecular property for the first molecule; and applying the trained molecular analysis model to determine the value of the molecular property for a second molecule.

In some variations of the methods, systems, and non-transitory computer readable media, one or more of the following features can optionally be included in any feasible combination.

In some variations, the training of the molecular analysis model may include minimizing a loss function quantifying a distance between two or more embeddings of augmented samples generated from a same conformer of the first molecule.

In some variations, the training of the molecular analysis model may exclude training the molecular analysis model to maximize a difference between two or more embeddings of augmented samples generated from different conformers of the first molecule.

In some variations, the training of the molecular analysis model may exclude training the molecular analysis model to maximize a difference between two or more embeddings of augmented samples generated from conformers of different molecules.

In some variations, the training of the molecular analysis model may include minimizing a loss function quantifying a difference between the value of the molecular property for the first molecule and a ground-truth value of the molecular property for the first molecule.

In some variations, the molecular analysis model may be trained to generate a second plurality of embeddings corresponding to a second plurality of augmented samples associated with a second conformer of the first molecule while minimizing a first difference between the second plurality of embeddings but not a second difference between the second plurality of embeddings and the first plurality of embeddings associated with the first conformer.

In some variations, the molecular analysis model may include a first machine learning model trained to generate the embedding for each augmented sample in the plurality of augmented samples.

In some variations, the molecular analysis model may further include a second machine learning model trained to determine, based at least on the embedding for each augmented sample, a respective value of the molecular property for each augmented sample.

In some variations, the molecular analysis model may determine, based at least on the respective value of the molecular property for each augmented sample, the value of the molecular property for the first molecule.

In some variations, the first plurality of augmented samples may include a first augmented sample having a first modification to the first three-dimensional structure of the first conformer and a second augmented sample having a second modification to the first three-dimensional structure of the first conformer.

In some variations, each of the first modification and the second modification may include a change to one or more of an atomic position, a bond angle, a bond length, and a dihedral angle present in the first three-dimensional structure of the first conformer.

In some variations, the change may include adding noise to the one or more of the atomic position, the bond angle, the bond length, and the dihedral angle present in the first three-dimensional structure of the first conformer.

In some variations, the first plurality of augmented samples may further include a third augmented sample having a third modification to the first three-dimensional structure of the first conformer.

In some variations, the molecular analysis model may be further trained to at least generate a third embedding of the third augmented sample while minimizing a difference between the third embedding and each of the first embedding and the second embedding, and determine, based at least on the third embedding, the value of the molecular property for the first molecule.

In some variations, the molecular analysis model may be trained to perform a classification task or a regression task in order to determine the value of the molecular property.

In some variations, the molecular property may include binding affinity, absorption, distribution, metabolism, potency, efficacy, phenotypic effects, or excretion.

In some variations, the trained molecular analysis model may determine the value of the molecular property of the second molecule by at least generating, for a second conformer of the second molecule, a first augmented sample and a second augmented sample by at least modifying a second three-dimensional structure of the second conformer, generating a first embedding for the first augmented sample and a second embedding for the second augmented sample, determining, based at least on the first embedding, the value of the molecular property for the first augmented sample, determining, based at least on the second embedding, the value of the molecular property for the second augmented sample, determining, based at least on the value of the molecular property for each of the first augmented sample and the second augmented sample, the value of the molecular property for the second conformer of the second molecule; and determining, based at least on the value of the molecular property for the second conformer, the value of the molecular property for the molecule.

In some variations, the first conformer of the first molecule may be selected from a conformer ensemble including a plurality of conformers associated with the first molecule. The plurality of conformers may have a same chemical composition but differ in structure via one or more rotations around intramolecular bonds.

In some variations, the molecular analysis model may be based at least on a subset of conformers comprising a random selection of conformers from a conformer ensemble of the first molecule.

Implementations of the current subject matter can include, but are not limited to, methods consistent with the descriptions provided herein as well as articles that comprise a tangibly embodied machine-readable medium operable to cause one or more machines (e.g., computers, etc.) to result in operations implementing one or more of the described features. Similarly, computer systems are also described that may include one or more processors and one or more memories coupled to the one or more processors. A memory, which can include a non-transitory computer-readable or machine-readable storage medium, may include, encode, store, or the like one or more programs that cause one or more processors to perform one or more of the operations described herein. Computer implemented methods consistent with one or more implementations of the current subject matter can be implemented by one or more data processors residing in a single computing system or multiple computing systems. Such multiple computing systems can be connected and can exchange data and/or commands or other instructions or the like via one or more connections, including, for example, to a connection over a network (e.g. the Internet, a wireless wide area network, a local area network, a wide area network, a wired network, or the like), via a direct connection between one or more of the multiple computing systems, etc.

The details of one or more variations of the subject matter described herein are set forth in the accompanying drawings and the description below. Other features and advantages of the subject matter described herein will be apparent from the description and drawings, and from the claims. While certain features of the currently disclosed subject matter are described for illustrative purposes in relation to Siamese networks trained to in a non-contrastive manner, it should be readily understood that such features are not intended to be limiting. The claims that follow this disclosure are intended to define the scope of the protected subject matter.

### DESCRIPTION OF DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1 depicts a system diagram illustrating an example of a molecular analysis system, in accordance with some example embodiments;

FIG. 2A depicts a flowchart illustrating an example of a process for molecular analysis, in accordance with some example embodiments;

FIG. 2B depicts a flowchart illustrating another example of a process for molecular analysis, in accordance with some example embodiments;

FIG. 3 depicts a schematic diagram illustrating an example of a molecular analysis pipeline, in accordance with some example embodiments;

FIG. 4 depicts graphs illustrating the training profiles of a molecular analysis model trained to perform a target task and an auxiliary task in a non-contrastive manner, in accordance with some example embodiments;

FIG. 5 depicts graphs illustrating the receiver operating characteristic area under the curve (ROCAUC) scores of a molecular analysis model trained to perform a target task and an auxiliary task in a non-contrastive manner, in accordance with some example embodiments;

FIG. 6 depicts graphs illustrating the manifold smoothness associated with an auxiliary task that a molecular analysis model is trained to perform in a non-contrastive manner, in accordance with some example embodiments;

FIG. 7 depicts graphs illustrating a degree of partial dimensional collapse associated with an auxiliary task that a molecular analysis model is trained to perform in a non-contrastive manner, in accordance with some example embodiments;

FIG. 8 depicts a block diagram illustrating an example of a computing system, in accordance with some example embodiments.

When practical, similar reference numbers denote similar structures, features, or elements.

### DETAILED DESCRIPTION

The molecular properties of a molecule may often be dependent on the three-dimensional structure of the molecule. For example, the binding affinity between a drug molecule and a target molecule (e.g., a protein, a nucleic acid, and/or the like) depends on the ability of the drug molecule to adopt a three-dimensional structure, or conformational shape, that is complementary to that of the target molecule. As such, for small molecules and large molecules alike, modeling the conformational shapes of a molecule may be critical in many molecular machine learning (MolML) tasks in which one or more machine learning models are trained to learn the relationship between molecular properties and conformational shapes. However, molecules tend to be flexible and can exist as an ensemble of conformations in equilibrium with one another. In the context of binding affinity, for instance, the biologically active conformation of a molecule may be one or more of the conformations exhibited by the molecule in solution or a new conformation that is induced by interaction with the target molecule. Nevertheless, many programs in machine learning-based drug discovery (MLDD) rely on small, noisy datasets (0(10e²⁻⁴)) containing complex molecular structures. As such, the development of machine learning models, such as three dimensional neural networks (NNs), that are capable of generalizing across a multitude of molecular geometries is particularly challenging.

In some example embodiments, a molecular analysis model may be trained to generalize across a multitude of molecular geometries such that the molecular analysis model is able to accurately determine one or more properties of a molecule without being confounded by minor variations in the three-dimensional structure (or conformation) of the molecule. For example, the molecular analysis model may be trained to generalize across different molecular geometries by at least training the molecular analysis model to perform an auxiliary task in which the molecular analysis model generates an embedding for each augmented sample formed by modifying the three-dimensional structure of at least one conformer of the molecule. In addition, the molecular analysis model may be trained to perform a target task in which the molecular analysis model determines, based at least on the embeddings, the value of a molecular property of the molecule such as, for example, binding affinity, absorption, distribution, metabolism, potency, efficacy, phenotypic effects, excretion, and/or the like. It should be appreciated that a single molecule may be associated with multiple conformers, which may be referred to collectively as a conformer ensemble (CE). For a single conformer of the molecule, multiple augmented samples may be generated by altering the three-dimensional structure of the conformer. As will be described in more detail below, the molecular analysis model may be trained to differentiate between individual conformers of the same molecule because different conformers of the same molecule may behave differently in biochemical systems despite similarities in three-dimensional structure. Moreover, the molecular analysis model may be trained to avoid unwarranted assumptions that chemically distinct molecules necessarily possess different properties. In fact, molecules with distinct chemical compositions, as reflected by unrelated two-dimensional connectivity graphs, may adopt very similar three-dimensional structures and exhibit similar properties.

As used herein, the terms “conformer” and “molecular conformer” may be used interchangeably to refer a molecule with a same chemical composition as another molecule (or conformer) but exhibiting one or more structural differences, for example, in atomic position (e.g., (x, y, z) coordinates of each constituent atom), bond length (e.g., distance between two connected atoms), bond angle (e.g., defined by two bonds connecting three atoms), dihedral angle (e.g., defined by half-planes through two sets of three atoms that share two atoms in common), and/or the like. That is, for the two molecules to be considered conformers (or molecular conformers) of one another, the difference in their respective three-dimensional structures may be reconciled to allow superimposition of the two molecules without breaking and reforming any intramolecular covalent bonds. Since the chemical composition of a molecule may be represented by a two-dimensional connectivity graph, the conformer ensemble (CE) of the molecule may include three-dimensional structures derived from the same two-dimensional connectivity graph. Moreover, in some cases, the three-dimensional structures in the conformer ensemble (CE) of the molecule may exhibit a geometric difference (e.g., pairwise root-mean-squared deviation (RMSD)) satisfying one or more thresholds (e.g., RMSD≥0.1 Å). In some cases, the term “molecule” may refer to the corresponding conformer ensemble (CE), which contains the various conformers of the molecule. However, since different conformers of the same molecule may exhibit different properties (or different values of a property), the property of the molecule may but is not necessarily the same as the property of any individual conformers of the molecule. Contrastingly, while the augmented samples of a conformer also exhibit one or more structural differences (e.g., atomic position, bond length, bond angle, dihedral angle, and/or the like), the augmented samples of a second conformer of a molecule may exhibit less structural differences than the augmented samples of a second conformer of the same molecule. For example, while the geometric difference (e.g., pairwise root-mean-squared deviation (RMSD)) between the first conformer and the second conformer may satisfy a first threshold (e.g., RMSD≥0.1 Å), the geometric difference (e.g., RMSD) between augmented samples of each of the first conformer and the second conformer may satisfy a second threshold (e.g., 0.05 Å t≤RMSD<0.1 Å).

In some example embodiments, the molecular analysis model may generate, for a conformer of the molecule, multiple augmented samples by at least applying one or more modifications to the three-dimensional structure of the conformer. Examples of modifications that may be applied to the three-dimensional structure of the conformer include changing the atomic position (e.g., (x, y, z) coordinates of the constituent atoms), bond length (e.g., defined by the distance between two connected atoms), bond angle (e.g., defined by two bonds connecting three atoms), dihedral angle (e.g., defined by half-planes through two sets of three atoms that share two atoms in common), and/or the like. In some cases, the one or more modifications may be achieved by applying noise (e.g., Gaussian noise) to the three-dimensional structure of the conformer. For example, in some cases, noise (e.g. Gaussian noise) may be applied to change one or more atomic positions, bond angles, and/or dihedral angles present in the three-dimensional structure of the conformer. As described in more detail below, the modifications to the three-dimensional structure of the conformer may be modulated in order to ensure that each augmented sample is probable (e.g., realistic and consistent with what is or expected to be observed in nature) and a threshold magnitude of geometric difference (e.g., a minimum RMSD and/or a maximum RMSD) exists between individual augmented samples of the conformer.

In some example embodiments, the molecular analysis model may include a first machine learning model trained to perform the auxiliary task of generating a first embedding for a first augmented sample having a first modification to the three-dimensional structure of the conformer and a second embedding for a second augmented sample having a second modification to the three-dimensional structure of the conformer. In some cases, each of the first modification and the second modification may include one or more changes to the atomic positions, bond angles, and/or dihedral angles present in the three-dimensional structure of the conformer. Moreover, the first augmented sample and the second augmented sample may exhibit a threshold level of structural similarities such that the first machine learning model may be trained to generate the first embedding of the first augmented sample to be similar to the second embedding of the second augmented sample. In some cases, the molecular analysis model may further include a second machine learning model trained to perform the target task of determining, based at least on the embedding of each augmented sample, a respective value of the molecular property for each augmented sample. Given the similarities between the first embedding of the first augmented sample and the second embedding of the second augmented sample, the second machine learning model may determine similar values for the molecular property of the first augmented sample and the second augmented sample. In some cases, the value of the molecular property of the molecule may be determined based at least on the value of the molecular property for multiple augmented samples of at least one conformer of the molecule.

In some example embodiments, the molecular analysis model may be trained in a non-contrastive manner to perform the auxiliary task of generating the embedding for each augmented sample. In some cases, the embedding of an augmented sample may be a latent representation (e.g., a latent vector and/or the like) of the augmented sample that represents the three-dimensional geometry of the augmented sample with a fewer quantity of features (or dimensions) than is present in the original feature space of the augmented sample. That is, the auxiliary task of generating the embedding for each augmented sample may include reducing the dimensionality of each augmented sample by at least mapping each augmented sample from the higher dimensional feature space to a lower dimensional latent space (e.g., a manifold and/or the like). Accordingly, the first machine learning model of the molecular analysis model may be trained to embed the augmented samples associated with the same conformer to latent representations (e.g., vector and/or the like) that occupy proximate positions in the latent space (e.g., a manifold and/or the like). That is, in some cases, the training of the molecular analysis model may include minimizing a difference (e.g., distance and/or the like) between the embeddings of the augmented samples generated by modifying the three-dimensional structure of the same conformer. Moreover, the training of the first machine learning model may exclude training the first machine learning model to embed dissimilar molecular geometries, such as augmented samples generated from different conformers of the same molecule or different molecules, to latent representation (e.g., vector and/or the like) that occupy proximate positions on the latent space (e.g., a manifold and/or the like).

Training the molecular analysis model in a non-contrastive manner may ensure that the first machine learning model generates similar embeddings for sufficiently similar molecular geometries, which is the case for augmented samples originating from the same conformer. However, the non-contrastive training also prevents the molecular analysis model from developing any bias towards generating dissimilar embeddings for dissimilar molecular geometries, such as augmented samples derived from different conformers of the same molecule and augmented samples derived from a different molecule. This behavior is consistent with the observation that different conformers of the same molecule may still behave differently in biochemical systems despite similarities in three-dimensional structure. Furthermore, this behavior is also consistent with the observation that molecules with different chemical compositions, as reflected by unrelated two-dimensional connectivity graphs, may still adopt similar three-dimensional structures and exhibit similar properties. Trained in a non-contrastive manner, the resulting molecular analysis model may exhibit a suitable level of sensitivity to changes in the composition of a molecule (e.g., as reflected in the corresponding two-dimensional connectivity graph) as well as a suitable level of insensitivity to changes in the three-dimensional structure of the molecule (e.g., atomic positions, bond length, bond angle, dihedral angle, and/or the like).

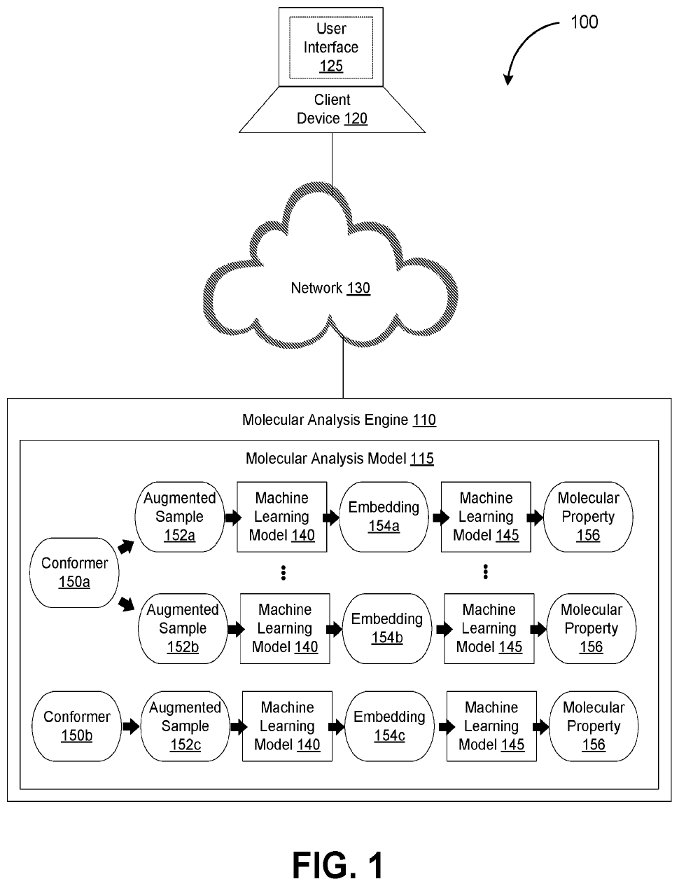

FIG. 1 depicts a system diagram illustrating an example of a molecular analysis system 100, in accordance with some example embodiments. Referring to FIG. 1 the molecular analysis system 110 may include a molecular analysis engine 110 and a client device 120 communicatively coupled via a network 130. The client device 120 may be a processor-based device including, for example, a workstation, a desktop computer, a laptop computer, a smartphone, a tablet computer, a wearable apparatus, and/or the like. The network 130 may be a wired network and/or a wireless network including, for example, a local area network (LAN), a virtual local area network (VLAN), a wide area network (WAN), a public land mobile network (PLMN), the Internet, and/or the like.

Referring again to FIG. 1, the molecular analysis engine 110 may train and apply a molecular analysis model 115 to determine the molecular property of a molecule including, for example, binding affinity, absorption, distribution, metabolism, potency, efficacy, phenotypic effects, excretion, and/or the like. The molecule may be a protein molecule or a non-protein molecule include small molecules, ions, nucleic acids, polysaccharides, glycolipids, and/or the like. As shown in FIG. 1, in some cases, the molecular analysis model 115 may include a first machine learning model 140 trained to perform the auxiliary task of generating an embedding for each augmented sample formed by modifying the three-dimensional structure of at least one conformer of the molecule. For example, the first machine learning model 140 may generate, for a first augmented sample 152a of a first conformer 150a of the molecule, a first embedding 154a. Moreover, the first machine learning model 140 may also generate, for a second augmented sample 152b of the first conformer 150a, a second embedding 154b. In some cases, the first augmented sample 152a and the second augmented sample 152b may be generated by an augmented sample generator 113 modifying the three-dimensional structure of the first conformer 150a. For instance, the first augmented sample 152a may include a first modification to the three-dimensional structure of the first conformer 150a while the second augmented sample 152b may include a second modification to the three-dimensional structure of the first conformer 150a. The first modification and the second modification may each include at least one change to an atomic position (e.g., the (x, y, z) coordinates of one or more constituent atoms), a bond length (e.g., defined by the distance between two connected atoms), a bond angle (e.g., defined by two bonds connecting three atoms), and/or a dihedral angle (e.g., defined by half-planes through two sets of three atoms having two atoms in common) present in the three-dimensional structure of the first conformer 150a. In some cases, the at least one change may be realized by the augmented sample generator 113 adding noise (e.g., Gaussian noise and/or the like) to an atomic position, a bond length, a bond angle, and/or a dihedral angle present in the three-dimensional structure of the first conformer 150a.

Referring again to FIG. 1, in some example embodiments, the molecular analysis model 115 may also include a second machine learning model 145 trained to perform the target task of determining, based at least on the embeddings, a value of the molecular property for the molecule. For example, FIG. 1 shows that the second machine learning model 145 may determine, based at least on the first embedding 154a of the first augmented sample 152a, the value of the molecular property 156 for the first augmented sample 152a. Furthermore, FIG. 1 shows that the second machine learning model 145 may determine, based at least on the second embedding 154b of the second augmented sample 152b, the value of the molecular property 156 for the second augmented sample 154b. In some cases, the molecular analysis model 115 may determine, based at least on the value of the molecular property 156 for each of the first augmented sample 152a and the second augmented sample 152b, the value of the molecular property 156 for the first conformer 150a (or the corresponding molecule). For instance, in some cases, the value of the molecular property 156 for the first conformer 150a may include a mean, a median, and/or a mode of the value of the molecular property 156 for each of the first augmented sample 152a and the second augmented sample 152b.

In some example embodiments, the modifications made to the three-dimensional structure of the first conformer 150a may be modulated in order to avoid generating improbable molecular geometries that are unlikely to exist in nature because such molecular geometries are inconsistent with what is or expected to be observed in nature. In some cases, an improbable molecular geometry may be an unrealistic molecular geometry whose likelihood of occurring in nature fails to satisfy one or more thresholds. Contrastingly, a probable molecular geometry may be a realistic molecular geometry whose likelihood of occurring in nature satisfies the one or more thresholds. Training the first machine learning model 140 and the second machine learning model 145 with improbable molecular geometries may impair the performance of each model. For example, avoiding improbable molecular geometries may prevent the first machine learning model 140 from being trained to generate, for an augmented sample having an improbable molecular geometry, a similar embedding as another augmented sample having a improbable molecular geometry. Further downstream, avoiding improbable geometries may prevent the second machine learning model 145 from being trained to determine similar property values for the similar embeddings of probable and improbable molecular geometries.

Accordingly, in some cases, the augmented sample generator 113 may modulate the type of modifications made to the three-dimensional structure of the first conformer 150a in order to ensure that the first augmented sample 152a and the second augmented sample 152b resulting therefrom are probable (e.g., realistic and consistent with what is or expected to be observed in nature). For example, in some cases, the first modification and the second modification may include changes (e.g., noise) applied to one or more bond lengths, bond angles, and/or dihedral angles present in the three-dimensional structure of the first conformer 150a but not atomic positions at least because changing atomic positions may yield improbable (or unrealistic) molecular geometries. Alternatively and/or additionally, the augmented sample generator 113 may modulate the extent of the modifications made to the three-dimensional structure of the first conformer 150a in order to achieve a threshold magnitude of geometric difference (e.g., a minimum root-mean-square deviation (RMSD) and/or a maximum RMSD) between the first conformer 150a and each of the first augmented sample 152a and the second augmented sample 152b. The threshold magnitude of geometric difference may be necessary in order to train the first machine learning model 140 to recognize when two different molecular geometries are sufficiently similar to merit similar embeddings. For instance, in some cases, the quantity (or scale) of noise added to modify the three-dimensional structure of the first conformer 150a may satisfy a first threshold (e.g., a minimum noise scale) such that the first augmented sample 152a and the second augmented sample 152b exhibit a sufficient magnitude of geometric difference relative to the first conformer 150a. In some cases, the quantity (or scale) of noise added to modify the three-dimensional structure of the first conformer 150a may further satisfy a second threshold (e.g., a maximum noise scale) to prevent the molecular geometries of the first augmented sample 152a and the second augmented sample 152b from deviating too far from that of the first conformer 150 so as to become a different conformer of the molecule altogether.

In some example embodiments, the molecular analysis model 115 may undergo non-contrastive training in which the first machine learning model 140 is trained to embed similar molecular geometries to latent representations that occupy proximate positions in the latent space. For instance, in the example shown in FIG. 1, the first machine learning model 140 may be trained to minimize the distance (e.g., in latent space) between the first embedding 154a and the second embedding 154b at least because the first augmented sample 152a and the second augmented sample 152b, which are generated from the same first conformer 150a, exhibit sufficiently similar molecular geometries. However, the training of the molecular analysis model 115 may exclude training the first machine learning model 140 to embed dissimilar molecular geometries to latent representations that occupy distant positions in the latent space. For instance in the embodiment shown in FIG. 1, the training of the first machine learning model 140 excludes training the first machine learning model 140 to maximize the distance between the first embedding 154a and the second embedding 154b, which originate from the first conformer 150a, and a third embedding 154c generated based on a third augmented sample 152c associated with a second conformer 150b, whether the second conformer 150b is associated with a same molecule or a different molecule as the first conformer 150a. Even in instances where the molecular geometry of the third augmented sample 152c is sufficiently different than that of the first augmented sample 152a and the second augmented sample 152b (e.g., RMSD≥0.1 Å) so as to constitute different conformers of the molecule, the first machine learning model 140 may not be trained to maximize the distance between the third embedding 154c and each of the first embedding 154a and the second embedding 154b.

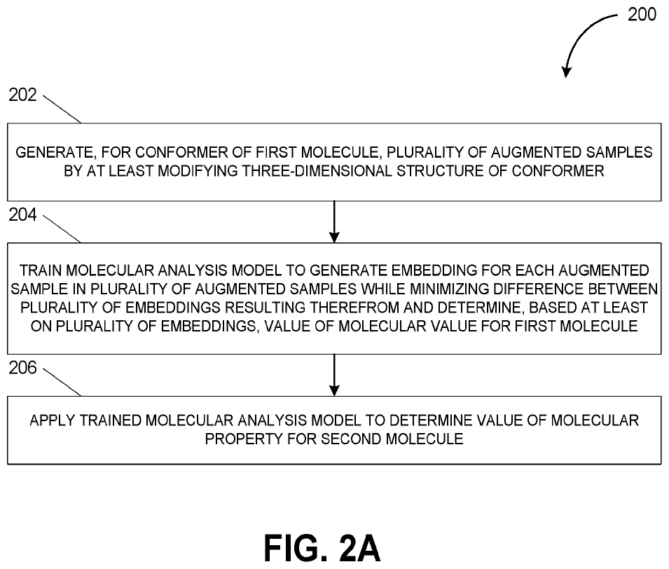

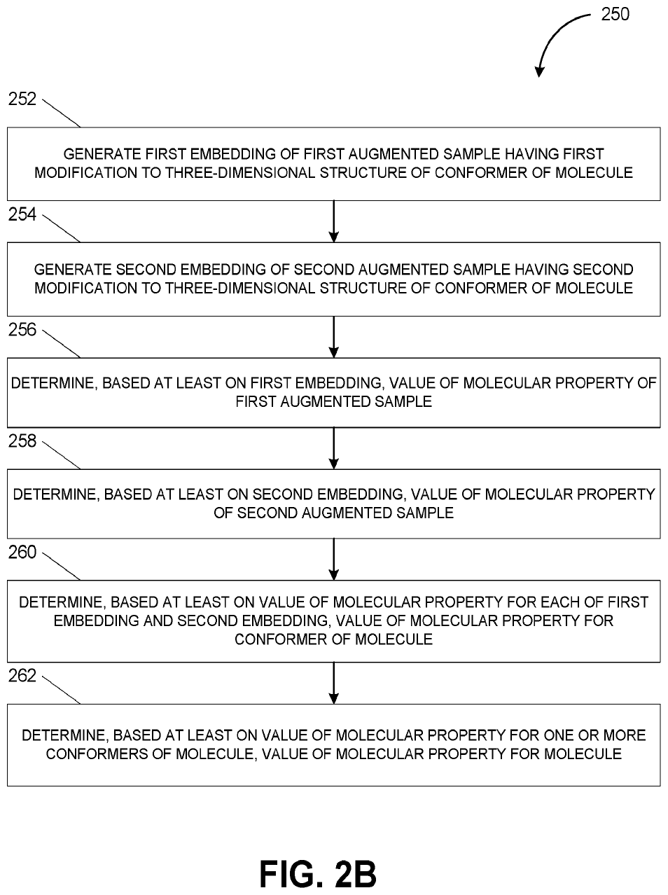

To further illustrate, FIG. 2A depicts a flowchart illustrating an example of a process 200 for molecular analysis, in accordance with some example embodiments. Referring to FIGS. 1 and 2A, the process 200 may be performed by the molecular analysis engine 110.

At 202, the molecular analysis engine 110 may generate, for a conformer of a first molecule, a plurality of augmented samples by at least modifying a three-dimensional structure of the conformer. For example, as shown in FIG. 1, the augmented sample generator 113 may generate, for the first conformer 150a, a plurality of augmented samples that includes the first augmented sample 152a, the second augmented sample 152b, and/or the like. In some example embodiments, the molecular analysis engine 110 may generate the first augmented sample 152a and the second augmented sample 152b by at least modifying the three-dimensional structure of the first conformer 150a. In some cases, the three-dimensional structure of the first conformer 150a may be modified by at least changing one or more atomic positions, bond length, bond angle, and dihedral angle present in the three-dimensional structure of the first conformer 150a. For instance, the position of one or more atoms in the three-dimensional structure of the first conformer 150a may be changed by adding noise to one or more coordinates (e.g., (x, y, z) coordinates) defining the position of each atom. That is, in some cases, the augmented sample of ĉ of the conformer c may generated by sampling the Gaussian noise N(0, 1)∈R^{n×3} around normalized atomic positions

$${n}_{i}^{c}$$

### ∈V_{c}.

In some example embodiments, the modifications made to the three-dimensional structure of the first conformer 150a may be modulated in order to ensure that the first augmented sample 152a and the second augmented sample 152b resulting therefrom exhibit probable (or realistic) molecular geometries for the training of the first machine learning model 140 and the second machine learning model 145. For example, in some cases, the augmented sample generator 113 may favor the types of changes (e.g., changes to bond length, bond angle, and dihedral angle) that yield probable (or realistic) molecular geometries and avoid those types of changes (e.g., changes to atomic positions) that yield improbable (or unrealistic) molecular geometries. Alternatively, the augmented sample generator 113 may impose certain thresholds on the magnitude of the changes (e.g., a maximum noise scale, a minimum noise scale, and/or the like) made to the three-dimensional structure of the first confirmer 150a in order to achieve a threshold magnitude of geometric difference (e.g., a minimum root-mean-square deviation (RMSD) and/or a maximum RMSD) between the first conformer 150a and each of the first augmented sample 152a and the second augmented sample 152b. In the previous example formulation in which the augmented sample of ĉ of the conformer c is generated by sampling the Gaussian noise N(0, 1)∈R^{n×3} around normalized atomic positions

$${n}_{i}^{c}$$

∈V_{c}, a noise scale corresponding to the magnitude of the positional change may be controlled by imposing a temperature hyperparameter τ. That is, the noise that is added to the coordinates (e.g., (x, y, z) coordinates) defining the position of the at least one atom in an augmented sample may be sampled from N(−τ, τ). A similar temperature hyperparameter τ can also be applied to limit the extent of change that can be made to bond length, bond angle, and/or dihedral angle. A certain cutoff radius (e.g., 4.0 Å) may also be imposed for constructing radial graphs, to which self-loops were added.

In some cases, the first conformer 150a may be a random selection from the conformer ensemble (CE) of the corresponding molecule in order to maximize conformer diversity in training the molecular analysis model 115 and isolate the effects of non-contrastive learning from a dependence on starting conformers. For example, in some cases, instead of training the molecular analysis model 115 based on entire conformer ensembles, the molecular analysis engine 110 may randomly sample a subset of conformers that include some but not all of the conformers in the conformer ensemble of each molecule (e.g., c∈Cₘ) for each training epoch that the molecular analysis model 115 undergoes. Doing so may expose the molecular analysis model 115 to roughly a Boltzmann-weighted distribution of different molecular geometries while also being more computationally efficient than modeling entire conformer ensembles. Alternatively, the molecular analysis engine 110 may select, from the conformer ensemble of each molecule, a subset of conformers (e.g., including the first conformer 150a) based on the energy of the individual conformers. For instance, in some cases, the subset of conformers may include the conformers in the conformer ensemble exhibiting a lower (or lowest) energy compared to other conformers in the conformer ensemble. In some cases, the subset of conformers may be weighted by a ground-state energy of the molecule, which may be the lowest permitted energy state of the molecule. Selecting the subset of conformers based on the energies of the individual conformer may be tantamount to explicitly sampling a Boltzmann-weighted distribution.

While it is possible for the conformers encountered by the molecular analysis model 115 during training to converge to a small number of locally optimal geometries, this bias may nevertheless be consistent with what is observed in a biological setting. In some cases, conformer diversity may be further imposed by ensuring that the conformers selected for training the molecular analysis model 115, such as the first conformer 150a, exhibit sufficient structural dissimilarities. For instance, in some cases, the first conformer 150a may be selected for training the molecular analysis model 115 if a dissimilarity metric (e.g., root mean square deviation (RMSD)) between the three-dimensional structure of the first conformer 150a and that of other conformers encountered by the molecular analysis model 115 satisfy one or more thresholds (e.g., RMSD≥0.1 Å).

At 204, the molecular analysis engine 110 may train the molecular analysis model 115 to generate an embedding for each augmented sample in the plurality of augmented samples while minimizing a difference between a plurality of embeddings resulting therefrom, and determine, based at least on the plurality of embeddings, a value of a molecular property for the first molecule. In some example embodiments, the molecular analysis engine 110 may train the molecular analysis model 115 to perform the auxiliary task of generating an embedding for each augmented sample associated with the first conformer 150a while minimizing the difference (e.g., distance and/or the like) between the resulting plurality of embeddings. Moreover, the molecular analysis engine 110 may train the molecular analysis model 115 to perform the target task of determining the value of the molecular property 156 for the first conformer 150a (or the corresponding molecule) based on the embeddings of the augmented samples associated with the first conformer 150a. For instance, in the example shown in FIG. 1, the molecular analysis engine 110 may train the first machine learning model 140 to perform the auxiliary task of generating the first embedding 154a of the first augmented sample 152a and the second embedding 154b of the second augmented sample 152b while minimizing a difference (e.g. distance and/or the like) between the first embedding 154a and the second embedding 154b. Furthermore, the molecular analysis engine 110 may train the second machine learning model 145 to perform the target task of determining, based at least on the first embedding 154a and the second embedding 154b, the value of the molecular property 156 for the first conformer 150a (or the corresponding molecule). For example, the training of the molecular analysis model 115 may include training the second machine learning model 145 to minimize a difference in the value of the molecular property 156 and the ground truth value of the molecular property 156.

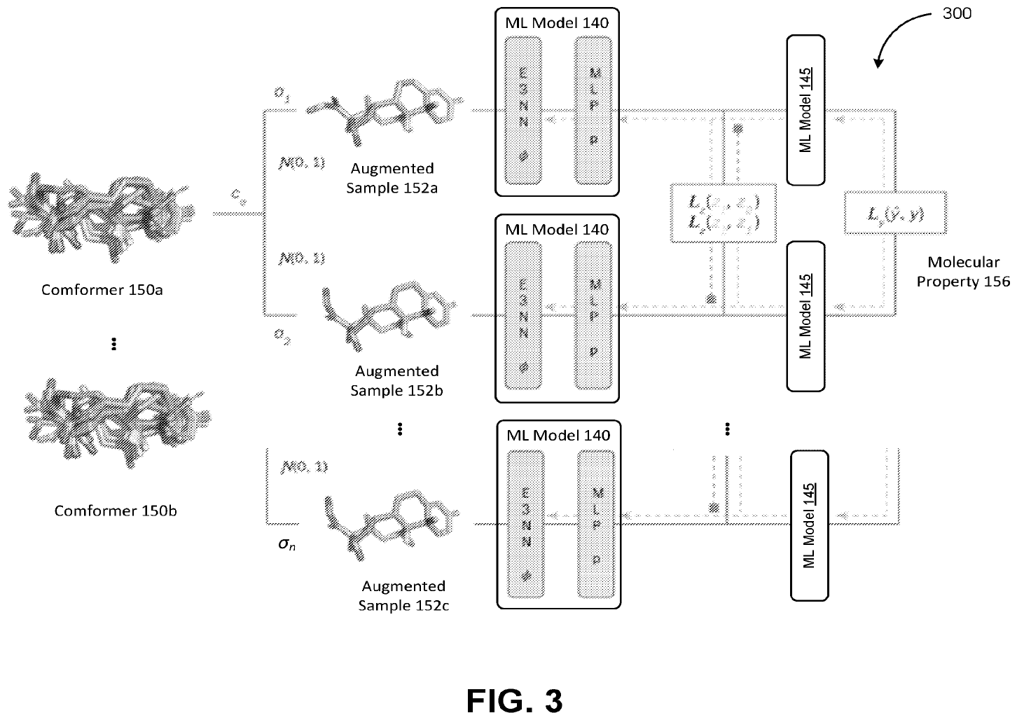

In some example embodiments, the molecular analysis model 115 may be trained in a non-contrastive manner, which includes training the first machine learning model 140 to minimize the difference (e.g., distance and/or the like) between embeddings of augmented samples derived from the same conformer such as, for example, the first embedding 154a of the first augmented sample 152a and the second embedding 154b of the second augmented sample 152b. Accordingly, the training of the molecular analysis model 115 may exclude training the first machine learning model 140 to maximize the difference (e.g., distance and/or the like) between embeddings of augmented samples derived from different conformers of the same molecule as well as embeddings of augmented samples derived from different molecules. As shown in FIGS. 1 and 3, for example, the training of the molecular analysis model 115 may exclude training the first machine learning model 140 to maximize the difference (e.g., distance and/or the like) between the first embedding 154a and the second embedding 154b associated with the first conformer 150a and the third embedding 154c generated based on the third augmented sample 152c of the second conformer 150b, whether the second conformer 150b is associated with a same molecule or a different molecule as the first conformer 150a.

At 206, the molecular analysis engine 110 may apply the trained molecular analysis model 115 to determine the value of the molecular property for a second molecule. In some example embodiments, the trained molecular analysis model 115 may be applied to determine the value of the molecular property 156 for one or more other molecules. Examples of the molecular property 156 may include binding affinity, absorption, distribution, metabolism, potency, efficacy, phenotypic effects, excretion, and/or the like. For example, in some cases, the trained molecular analysis model 115 may be applied to perform a classification task that includes assigning one or more discrete labels to a molecule to indicate whether the molecule exhibits the molecular property 156 (e.g., a binary label indicative of a binder and a non-binder). Alternatively and/or additionally, the trained molecular analysis model 115 may be applied to perform a regression task that includes assigning one or more continuous labels to indicate the magnitude (or degree) of the molecular property 156 exhibited by the molecule.

To further illustrate the training of the molecular analysis model 115 described in operation 204 of the process 200, FIG. 3 depicts a schematic diagram illustrating an example of a molecular analysis pipeline 300 associated with the molecular analysis model 115, in accordance with some example embodiments. As shown in FIG. 3, the first machine learning model 140 may be applied to generate the first embedding 154a for the first augmented sample 152a of the first conformer 150a and the second embedding 154b for the second augmented sample 154b of the first conformer 150a. Moreover, the second machine learning model 145 may be applied to determine the value of the molecular property 156 for the first augmented sample 152a based on the first embedding 154a and the value of the molecular property 156 for the second augmented sample 152b based on the second embedding 154b. The molecular analysis pipeline 300 shown in FIG. 3 may be associated with an overall loss L expressed by Equations (1) and (2) below. According to Equations (1) and (2), the overall loss L of the molecular analysis pipeline 300 may include a target prediction loss term L_{y} corresponding to the loss associated with the value of the molecular property 156, an embedding loss Lₛ associated with the difference between the embeddings of augmented samples, and an L2 regularization penalty Lᵣ.

$$L = \frac{1}{N} {\sum }_{i = 1}^{N} ( \frac{1}{{C}_{m}} {\sum }_{j = 1}^{{C}_{m}} [ \frac{1}{A} {\sum }_{a = 1}^{A} ( {λ}_{y} {L}_{y} ( {\hat{y}}_{a}^{c} , {y}_{i} ) + {λ}_{r} {L}_{r} ( {z}_{a}^{c} ) + \frac{1}{A - 1} {\sum }_{a = 2}^{A} {λ}_{s} {L}_{s} ( {z}_{1}^{c} , {z}_{a}^{c} ) ) ] ) , \qquad ( 1 )$$

$${L}_{s} ( {z}_{1}^{c} , {z}_{a \neq 1}^{c} ) = - [ \frac{{z}_{1}^{c}}{{‖ {z}_{1}^{c} ‖}_{2}} , \frac{ξ ( {z}_{a}^{c} )}{{‖ {z}_{a}^{c} ‖}_{2}} ] - [ \frac{{z}_{a}^{c}}{{‖ {z}_{a}^{c} ‖}_{2}} , \frac{ξ ( {z}_{1}^{c} )}{{‖ {z}_{1}^{c} ‖}_{2}} ] ; {L}_{r} ( {z}_{a}^{c} ) = {‖ {z}_{a}^{c} ‖}_{2} \qquad ( 2 )$$

wherein N is the dataset size, Cₘ is the number of conformers of molecule m modeled in each pass of the molecular analysis pipeline 300, λₜ are subtask weights, A is the number of augmented samples modeled for each conformer,

$${z}_{1}^{c}$$

is the learned embedding of the parent,

$${z}_{a}^{c}$$

is the learned embedding of the augmented sample, yᵢ and

$${\hat{y}}_{a}^{c}$$

are the ground truth and predicted labels for the molecule i and the augmented conformer

$${\hat{c}}_{a}^{i} ,$$

respectively, and ξ(·) represents the stop gradient (stopgrad) operation that will be explained in more detail below.

In the example of the molecular analysis pipeline 300 shown in FIG. 3, the first machine learning model 140 may include a Euclidean neural network (E3NN) (or another equivariant or non-equivariant neural network) coupled with a readout multilayer perceptron (MLP). For example, in some cases, the trunk of each Euclidean neural network (E3NN) may include one or more convolutional interaction blocks, which are followed by global mean pooling over node features and the readout multi-layer perceptron (MLP). In some cases, a normalization layer may be applied to each convolution interaction block with the intermediate representations being batch normalized. The resulting parent and augmented representations

$${z}_{1}^{c} and {\hat{z}}_{a}^{c}$$

may be projected by the multilayer perceptron to give

$${z}_{1}^{c} and {z}_{a}^{c} .$$

Furthermore, in the example of the molecular analysis pipeline 300 shown in FIG. 3, the second machine learning model 145 is another multilayer perceptron (MLP). It should be appreciated that the first machine learning model 140 and the second machine learning model 145 may be implemented using different architectures than shown.

In some example embodiments, the aforementioned stop gradient (stopgrad) operation may be performed during the training of the molecular analysis model 115 to avoid the phenomenon of trivial collapse where the embeddings generated by the first machine learning model 140 collapse to a single trivial constant solution and that of partial dimensional collapse where the first machine learning model 140 generates embeddings that span a lower-dimensional subspace instead of the entire available latent space. That is, the stop gradient (stopgrad) operation may be performed to prevent the first machine learning model 140 from learning to generate the same embedding or the same set of embeddings for every input. For example, in some cases, the stop gradient (stopgrad) operation may include backpropagating the gradients of each augmented sample individually with the loss being symmetrized by multiple backward passes rotating the augmented samples. Referring again to Equation (1), the embedding loss Lₛ may translate to the first machine learning model 140 predicting the learned embedding

$${z}_{a}^{c}$$

of the augmented sample from the learned embedding of the parent

$${\hat{z}}_{1}^{c}$$

and vice versa. With the stop gradient (stopgrad) operation, each backward pass propagates the loss associated with a single augmented sample

$$( {z}_{a}^{c} ) ,$$

with the corresponding gradients detached from those of the remaining augmented samples

$${z}_{a}^{c} \neq i .$$

This is symmetrized such that each augmented sample α∈A receives a backward pass.

Referring again to FIG. 3, for the target task of determining the value of the molecular property 156 of the first conformer 150a (or the corresponding molecule), which is associated with the loss L_{y} in Equation (1), probabilistic inference may be utilized to account for aleatoric uncertainty in datasets. Accordingly, in some cases, the molecular analysis model 115 may output a probability distribution (e.g., a parameterized distribution over logits) to indicate across the possible values of the molecular property 156 for each conformer, from which sampling is performed prior to appropriate activation and loss calculation.

FIG. 2B depicts a flowchart illustrating another example of a process 250 for molecular analysis, in accordance with some example embodiments. Referring to FIGS. 1 and 2A-B, the process 250 may be performed when the molecular analysis engine 110 applies the trained molecular analysis model 115. In some cases, the process 250 may implement operation 206 of the process 200.

At 252, the molecular analysis model 115 may generate a first embedding of a first augmented sample having a first modification to a three-dimensional structure of a conformer of a molecule. For example, referring again to FIG. 1, in an inference setting where the trained molecular analysis model 115 is deployed to determine an unknown value of molecular property 156 of a molecule, the first machine learning model 140 of the molecular analysis model 115 may generate the first embedding 154a of the first augmented sample 152a of the first conformer 150a of the molecule.

At 254, the molecular analysis model 115 may determine, based at least on the first embedding, a value of a molecular property of the first augmented sample. For example, as shown in FIG. 1, in the inference setting, the second machine learning model 145 may generate, based at least on the first embedding 154a of the first augmented sample 152a, the value of the molecular property 156 for the first augmented sample 152a.

At 256, the molecular analysis model 115 may generate a second embedding of a second augmented sample having a second modification to the three-dimensional structure of the conformer of the molecule. For example, in addition to the first embedding 154a of the first augmented sample 152a, the first machine learning model 140 of the molecular analysis model 115 may also generate the second embedding 154b of the second augmented sample 152b of the first conformer 150a of the molecule.

At 258, the molecular analysis model 115 may determine, based at least on the second embedding, the value of the molecular property of the second augmented sample. For example, as shown in FIG. 1, in the inference setting, the second machine learning model 145 may generate, based at least on the second embedding 154b of the second augmented sample 152b, the value of the molecular property 156 for the second augmented sample 152b.

At 260, the molecular analysis model 115 may determine, based at least on the value of the molecular property for each of the first augmented sample and the second augmented sample, the value of the molecular property for the conformer of the molecule. In some example embodiments, the value of the molecular property 156 for the molecule (or the first conformer 150a of the molecule) may be determined based on the value of the molecular property 156 for each of the first augmented sample 152a and the second augmented sample 152b. For example, in some cases, the value of the molecular property 156 of the molecule (or the first conformer 150a of the molecule) may correspond to a mean, a mode, and/or a median of the respective values of the molecular property 156 for each of the first augmented sample 152a and the second augmented sample 152b.

At 262, the molecular analysis engine 110 may determine, based at least on the value of the molecular property for one or more conformers of the molecule, the value of the molecular property for the molecule. In some example embodiments, the molecular analysis engine 110 may determine the value of the molecular property 156 for the molecule based on the value of the molecular property 156 for a single conformer of the molecule such as the first conformer 150a. Alternatively, in some cases, the molecular analysis engine 110 may apply the molecular analysis model 115 to determine the value of the molecular property 156 for multiple conformers of the molecule (e.g., a threshold quantity of conformers of the molecule) including, for example, the first conformer 150a, the second conformer 150b, and/or the like. The value of the molecular property 156 of the molecule may be determined based on the value of the molecular property 156 of multiple individual conformers. For instance, in some cases, the value of the molecular property 156 for the molecule may be a mean, a median, and/or a mode of the respective values of the molecular property 156 for each of the individual conformers including the first conformer 150a, the second conformer 150b, and/or the like. In some cases, the three-dimensional structure of the molecule may be determined based at least on the value of the molecular property 156. Alternatively and/or additionally, the composition and/or the three-dimensional structure of the molecule may undergo modification based at least on the value of the molecular property 156. In some cases, where the value of the molecular property 156 of the molecule satisfies one or more thresholds, one or more additional molecules may be generated based on the composition and/or three-dimensional structure of the molecule.

In some example embodiments, training the molecular analysis model 115 in a non-contrastive manner, particularly with respect to the auxiliary task of generating embeddings, may result in more generalizable molecular analysis model 115 in small-data regimes. The ability of the trained molecular analysis model 115 to perform well when applied to data not encountered during training may be analyzed by quantifying local manifold smoothness (MS, η_{f}) as a proxy for the model's robustness to conformer noise in unseen data. It should be appreciated that in some cases, local manifold smoothness η(f, c) of the molecular analysis model f may be defined as the percentage of augmented samples cₐ from the input conformer c assigned the mode predicted label in the set. As shown in Equation (3), this formulation may be generalized to a probabilistic and regression setting by computing the divergence (e.g., Kullback-Leibler (KL) divergence) between the predicted posteriors (\hat{μ}_{c}, \hat{σ}_{c}) and (\hat{μ}ₐ, \hat{σ}ₐ) of the parent (e.g., the conformer c) and the augmented samples cₐ, respectively. In some cases, the value of η_{f} computed as such may be used to compare between variations of the molecular analysis model 115 with different subtask weights λₜ.

$${η}_{f} = \frac{1}{N} {\sum }_{n = 1}^{N} \frac{1}{A \cdot {C}_{m}} {\sum }_{i = 1}^{{C}_{m}} {\sum }_{a = 1}^{A} 1 - [ \text{} log log ( {\hat{σ}}_{a} ) - log log ( {\hat{σ}}_{c} ) + \frac{{( {\hat{σ}}_{a} )}^{2} + {( {\hat{μ}}_{a} - {\hat{μ}}_{c} )}^{2}}{2 {( {\hat{σ}}_{a} )}^{2}} - 0.5 ] \qquad ( 3 )$$

In some example embodiments, the phenomenon of trivial collapse where the molecular analysis model 115 learns a single trivial solution for every input may be detected by quantifying the variance in embeddings

$$( {σ}_{z}^{2} )$$

along uit feature axis. Meanwinle, the phenomenon of partial dimensional collapse in which the molecular analysis model 115 learns a limited set of solutions for every input may be detected by an analysis of the cumulative explained variance (CEV, Γ) of the singular values γ computed through principal component analysis (PCA) of embedding features. The cumulative explained variance up to rank-sorted γⱼ(Γⱼ) and the area under the full cumulative explained variance (CEV) curve (Γ) may be defined by Equation (4) below.

$${Γ}_{j} = \frac{{\sum }_{i = 1}^{j} {γ}_{i}}{{\sum }_{k = 1}^{d} {γ}_{k}} ; Γ = \frac{1}{d} {\sum }_{i = 1}^{d} {Γ}_{i} \qquad ( 4 )$$

wherein d is the full embedding size. In some cases, Γ may range between [0.5, 1.0], with larger values corresponding to more rapid coverage of the overall cumulative explained variance (CEV) over fewer singular values, and thus indicating a larger degree of partial dimensional collapse. Meanwhile, instances where Γ=0.5 correspond to zero partial dimensionality collapse.

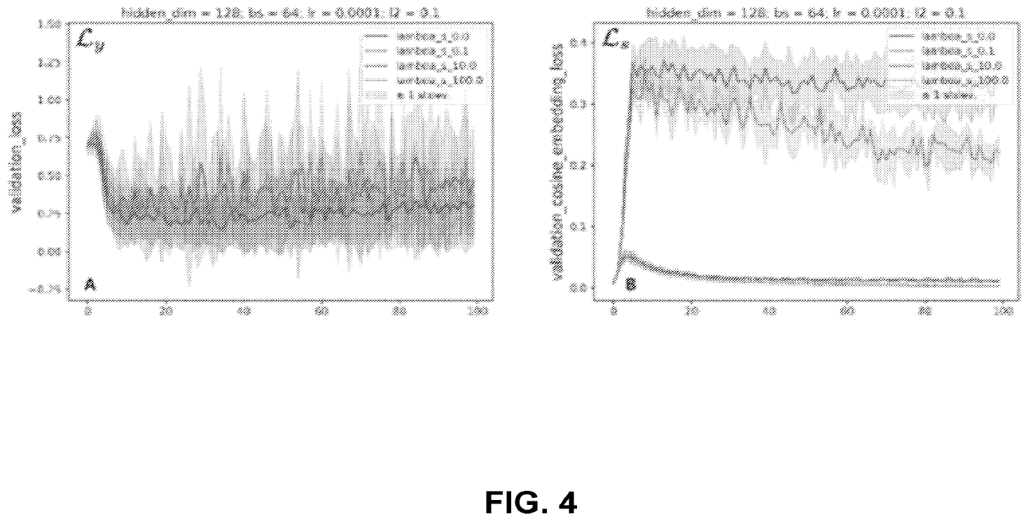

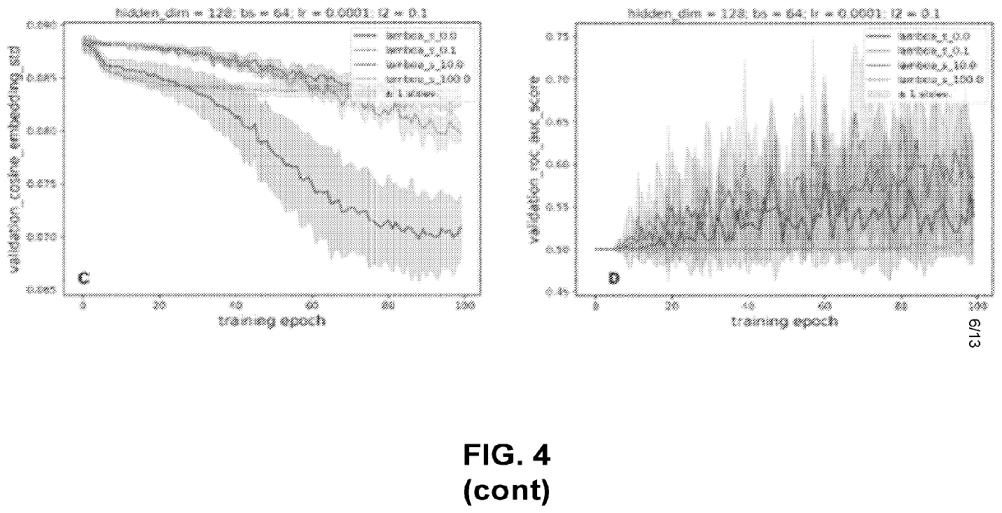

FIG. 4 depicts the training profiles of the first machine learning model 140 (e.g., the Euclidean neural network (E3NN)) for the classification task (e.g., the binary classification task of binding prediction) at various subtask weight values λₛ with λ=0.1. In a purely supervised training setting (e.g., λ=0), training curves are strikingly erratic across hyperparameter settings (FIG. 4A). Furthermore, cosine embedding distance for augmented samples remains high over the course of training (FIG. 4B). Meanwhile, latent feature variance decreases monotonically in some cases and in many cases is to a lesser degree than with λₛ>0 (FIG. 4C). Despite all this, the benchmark metric (ROC AUC score) increases throughout training (FIG. 4D) and converges to state of the art performance levels.

These training behaviors are markedly different with inclusion of the auxiliary task. Smoother loss curves are seen under many (but, importantly, not all) hyperparameter settings (FIG. 4A), particularly with λₛ≥1. A smooth reduction of the cosine embedding distance toward 0 is observed when the training for the auxiliary task is performed in a non-contrastive manner, with a trend at increasing λₛ (FIG. 4B). Variance in embedding features smoothly decreases at most values of λₛ (FIG. 4C). Finally, convergence of the benchmark metric (ROC AUC score) can be maintained at lower λₛ (FIG. 4D) and reaches state of art performance levels under many settings.

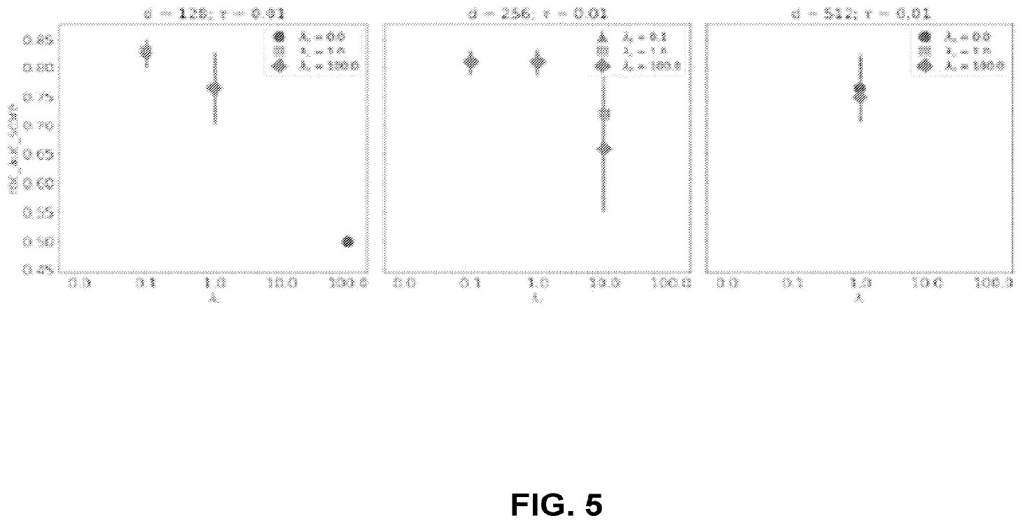

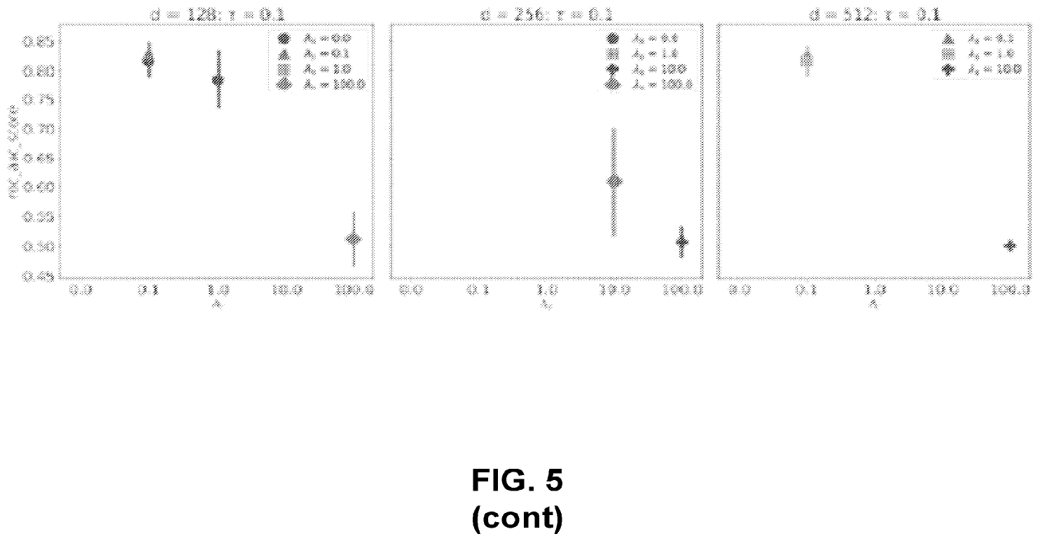

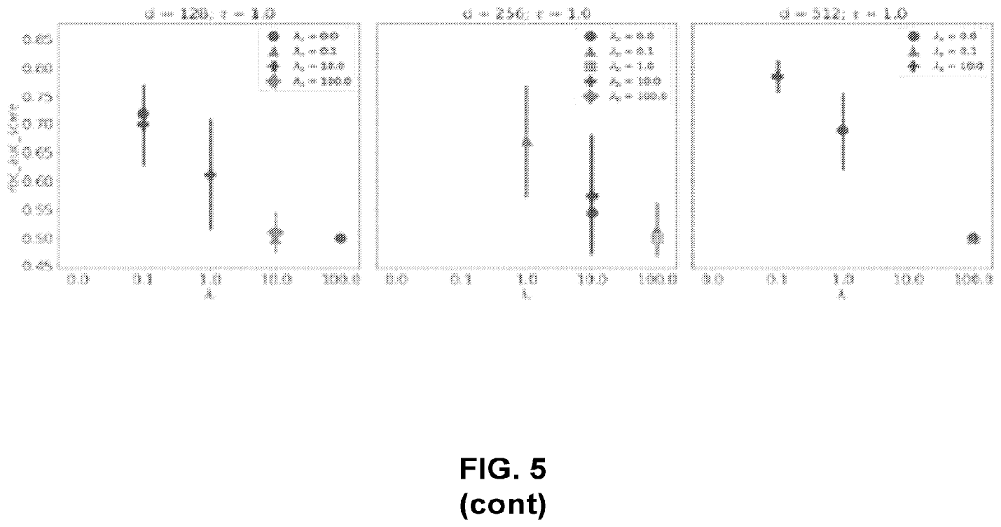

As shown in FIG. 5, increasing the subtask weight λᵣ past a critical point has a uniformly deleterious effect on the training and performance of the molecular analysis model 115. There are occasional similarities in the effects of the subtasks weights λᵣ and λₛ on the embedding loss Lₛ and

$${σ}_{2}^{z} .$$

Like with the subtak weight λₛ for the auxiliary embedding task, embedding loss Lₛ more rapidly converges at increasing subtask weight λᵣ for the target task, even with λₛ=0.

The performance of the molecular analysis model 115 on test sets are consistent with the training profiles, with test set area under the receiving operating characteristic curve (ROCAUC) is maintained with increasing embedding task weight λₛ while target prediction task weight λᵣ=0. When the target prediction task weight λᵣ is set higher, increasing the embedding task weight λₛ can be deleterious. At λᵣ≥1, the target task is no longer learned, regardless of the value of the embedding task weight λₛ. In general, performance has an increased dependence on hidden dimensions d of the embeddings generated by the first machine learning model 140 at higher embedding task weight λₛ, and vice versa.

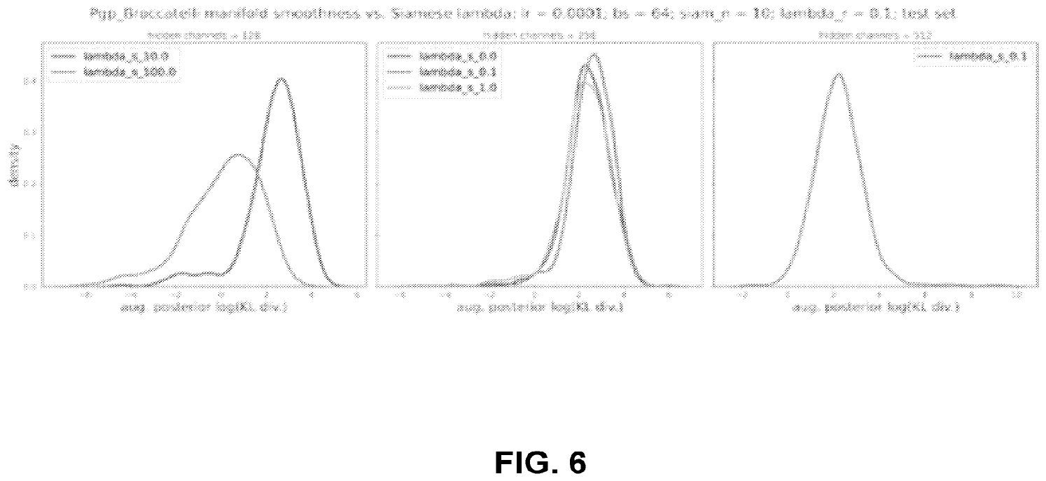

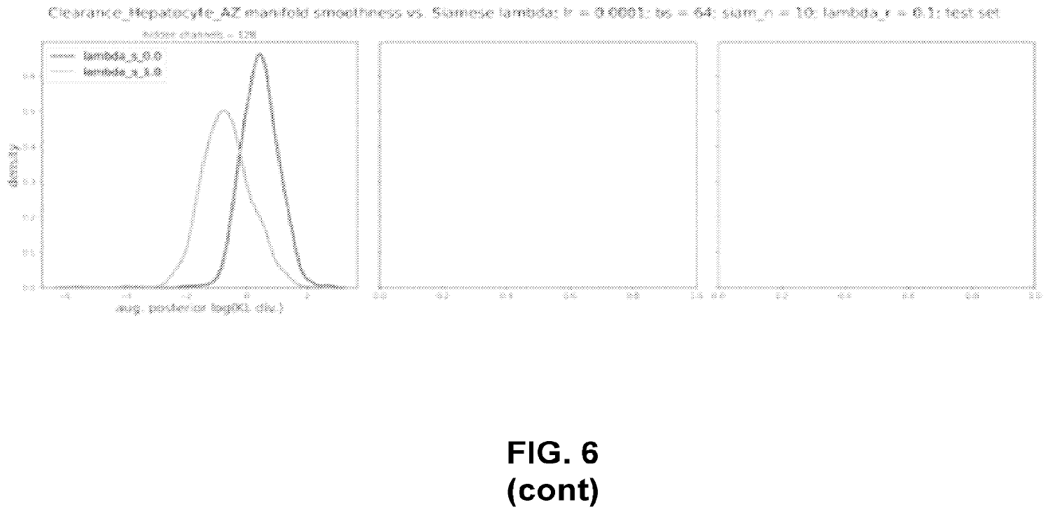

In some cases, manifold smoothness and partial dimensional collapse may be evaluated in order to determine whether the foregoing training profiles lead to greater generalization by the molecular analysis model 115. FIG. 6 shows the manifold smoothness associated with the training profiles shown in FIG. 4. As shown in FIG. 4, at larger hidden dimensions d, distributions of log log(KL) are largely indistinguishable (FIG. 4B,C). However, at d=128, distribution modes do indicate up to 1-3 log unit reduction in Kullback-Leibler divergence at λₛ≥1 (FIG. 4A). The absolute scale of Kullback-Leibler divergence reduces drastically at increasing subtask weights λᵣ for the target prediction task to indicate latent space compactification at high subtask weights λₛᵣ. The trends across λₛ remain largely unchanged.

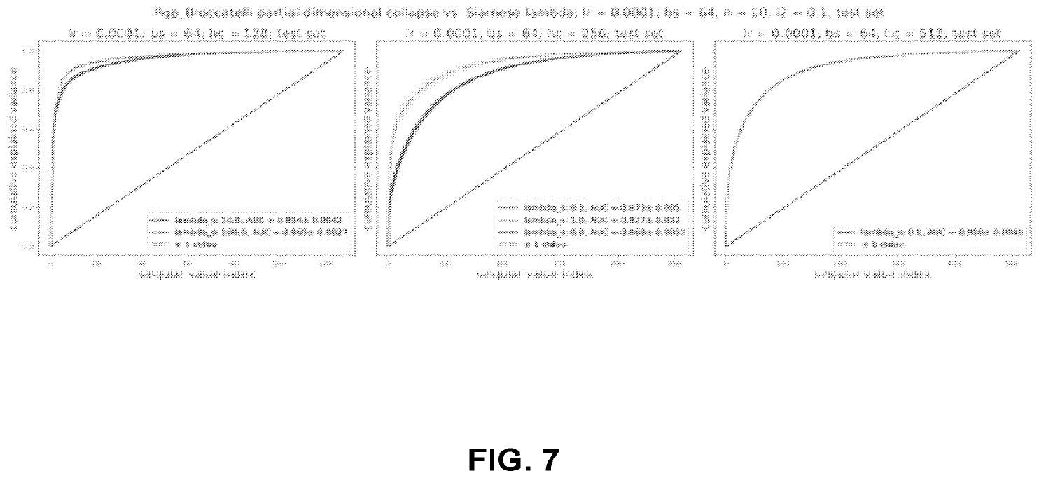

FIG. 7 shows the cumulative explained variance (CEV, Γ) for the molecular analysis model 115, which indicate a positive correlation between the area under the cumulative explained variance curve Γ and the embedding subtask weight λₛ. That said, at λₛ<1.0, no increase in partial dimensional collapse was observed (FIG. 5B). Also, a negative correlation between cumulative explained variance Γ and hidden dimensions d is observed up to an intermediate value of d, at which the correlation becomes positive. This observation indicates that for certain data settings (e.g., N˜10^{2_4}), medium-sized models may be of sufficient capacity, and thus no more information is encoded in latent vectors at increasing hidden dimensions d and higher area under the cumulative explained variance curve Γs are observed.

In view of the above-described implementations of subject matter this application discloses the following list of examples, wherein one feature of an example in isolation or more than one feature of said example taken in combination and, optionally, in combination with one or more features of one or more further examples are further examples also falling within the disclosure of this application:
Item 1: A computer-implemented method, comprising: generating, for a first conformer of a first molecule, a first plurality of augmented samples by at least modifying a first three-dimensional structure of the first conformer; training a molecular analysis model to generate an embedding for each augmented sample in the first plurality of augmented samples while minimizing a difference between a first plurality of embeddings resulting therefrom, and determine, based at least on the first plurality of embeddings, a value of a molecular property for the first molecule; and applying the trained molecular analysis model to determine the value of the molecular property for a second molecule.
Item 2: The method of Item 1, wherein the training of the molecular analysis model includes minimizing a loss function quantifying a distance between two or more embeddings of augmented samples generated from a same conformer of the first molecule.
Item 3: The method of any of Items 1 to 2, wherein the training of the molecular analysis model excludes training the molecular analysis model to maximize a difference between two or more embeddings of augmented samples generated from different conformers of the first molecule.
Item 4: The method of any of Items 1 to 3, wherein the training of the molecular analysis model excludes training the molecular analysis model to maximize a difference between two or more embeddings of augmented samples generated from conformers of different molecules.
Item 5: The method of any of Items 1 to 4, wherein the training of the molecular analysis model includes minimizing a loss function quantifying a difference between the value of the molecular property for the first molecule and a ground-truth value of the molecular property for the first molecule.
Item 6: The method of any of Items 1 to 5, further comprising: training the molecular analysis model to generate a second plurality of embeddings corresponding to a second plurality of augmented samples associated with a second conformer of the first molecule while minimizing a first difference between the second plurality of embeddings but not a second difference between the second plurality of embeddings and the first plurality of embeddings associated with the first conformer.
Item 7: The method of any of Items 1 to 6, wherein the molecular analysis model includes a first machine learning model trained to generate the embedding for each augmented sample in the plurality of augmented samples.
Item 8: The method of Item 7, wherein the first machine learning model includes an equivariant neural network coupled with a readout multilayer perceptron (MLP).
Item 9: The method of any of Items 7 to 8, wherein the molecular analysis model further includes a second machine learning model trained to determine, based at least on the embedding for each augmented sample, a respective value of the molecular property for each augmented sample.
Item 10: The method of Item 9, wherein the molecular analysis model determines, based at least on the respective value of the molecular property for each augmented sample, the value of the molecular property for the first molecule.
Item 11: The method of any of Items 9 to 10, wherein the value of the molecular property for the first molecule is a mean, a median, or a mode of the respective values of the molecular property for each augmented sample.
Item 12: The method of any of Items 9 to 11, wherein the second machine learning model is an artificial neural network.
Item 13: The method of any of Items 1 to 12, wherein the first plurality of augmented samples includes a first augmented sample having a first modification to the first three-dimensional structure of the first conformer and a second augmented sample having a second modification to the first three-dimensional structure of the first conformer.
Item 14: The method of Item 13, wherein the molecular analysis model is trained to at least generate a first embedding of the first augmented sample and a second embedding of the second augmented sample while minimizing a difference between the first embedding and the second embedding, and determine, based at least on the first embedding and the second embedding, the value of the molecular property for the first molecule.
Item 15: The method of Item 14, wherein the training of the molecular analysis model includes adjusting one or more weights of the molecular analysis model to minimize a first error associated the first embedding before further adjusting the one or more weights of the molecular analysis model to minimize a second error associated with the second embedding.
Item 16: The method of any of Items 14 to 15, wherein each of the first modification and the second modification include a change to one or more of an atomic position, a bond angle, a bond length, and a dihedral angle present in the first three-dimensional structure of the first conformer.
Item 17: The method of Item 16, wherein the change includes adding noise to the one or more of the atomic position, the bond angle, the bond length, and the dihedral angle present in the first three-dimensional structure of the first conformer.
Item 18: The method of any of Items 14 to 17, wherein the first plurality of augmented samples further include a third augmented sample having a third modification to the first three-dimensional structure of the first conformer.
Item 19: The method of Item 18, wherein the molecular analysis model is further trained to at least generate a third embedding of the third augmented sample while minimizing a difference between the third embedding and each of the first embedding and the second embedding, and determine, based at least on the third embedding, the value of the molecular property for the first molecule.
Item 20: The method of any of Items 1 to 19, wherein the molecular analysis model is trained to perform a classification task or a regression task in order to determine the value of the molecular property.
Item 21: The method of any of Items 1 to 20, wherein the molecular property includes binding affinity, absorption, distribution, metabolism, potency, efficacy, phenotypic effects, or excretion.
Item 22: The method of any of Items 1 to 21, wherein the second molecule is a protein molecule, a small molecule, an ion, a nucleic acid, a polysaccharide, or a glycolipid.
Item 23: The method of any of Items 1 to 22, further comprising: determining, based at least on the value of the molecular property of the second molecule, a second three-dimensional structure of the second molecule.
Item 24: The method of any of Items 1 to 23, further comprising: modifying, based at least on the value of the molecular property of the second molecule, a composition and/or a second three-dimensional structure of the second molecule.
Item 25: The method of any of Items 1 to 24, further comprising: generating, based at least on the value of the molecular property of the second molecule, a third molecule based at least on a composition and/or a second three-dimensional structure of the second molecule.
Item 26: The method of any of Items 1 to 25, wherein the trained molecular analysis model determines the value of the molecular property of the second molecule by at least generating, for a second conformer of the second molecule, a first augmented sample and a second augmented sample by at least modifying a second three-dimensional structure of the second conformer, generating a first embedding for the first augmented sample and a second embedding for the second augmented sample, determining, based at least on the first embedding, the value of the molecular property for the first augmented sample, determining, based at least on the second embedding, the value of the molecular property for the second augmented sample, determining, based at least on the value of the molecular property for each of the first augmented sample and the second augmented sample, the value of the molecular property for the second conformer of the second molecule; and determining, based at least on the value of the molecular property for the second conformer, the value of the molecular property for the molecule.
Item 27: The method of Item 26, wherein the value of the molecular property for the molecule is further determined based on the value of the molecular property for a third conformer of the second molecule.
Item 28: The method of any of Items 1 to 27, wherein the first conformer of the first molecule is selected from a conformer ensemble including a plurality of conformers associated with the first molecule, and wherein the plurality of conformers have a same chemical composition but differ in structure via one or more rotations around intramolecular bonds.
Item 29: The method of any of Items 1 to 28, further comprising: training the molecular analysis model based at least on a subset of conformers from a conformer ensemble of the first molecule.
Item 30: The method of Item 29, wherein the subset of conformers comprises a random selection of conformers from the conformer ensemble.
Item 31: A system, comprising: at least one data processor; and at least one memory storing instructions, which when executed by the at least one data processor, result in operations comprising the method of any of Items 1 to 30.
Item 32: A non-transitory computer readable medium storing instructions, which when executed by at least one data processor, result in operations comprising the method of any of Items 1 to 30.

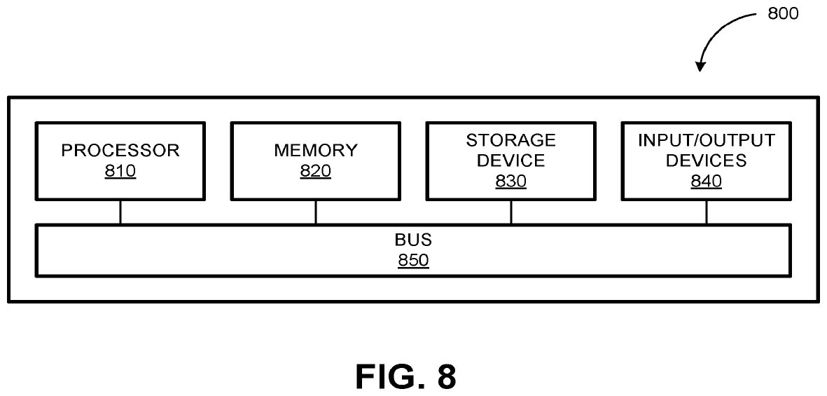

FIG. 8 depicts a block diagram illustrating an example of a computing system 800, in accordance with some example embodiments. Referring to FIGS. 1-8, the computing system 800 may be used to implement the molecular analysis engine 110, the client device 120, and/or any components therein.

As shown in FIG. 8, the computing system 800 can include a processor 810, a memory 820, a storage device 830, and input/output devices 840. The processor 810, the memory 820, the storage device 830, and the input/output devices 840 can be interconnected via a system bus 850. The processor 810 is capable of processing instructions for execution within the computing system 800. Such executed instructions can implement one or more components of, for example, the molecular analysis engine 110, the client device 120, and/or the like. In some example embodiments, the processor 810 can be a single-threaded processor. Alternately, the processor 810 can be a multi-threaded processor. The processor 810 is capable of processing instructions stored in the memory 820 and/or on the storage device 830 to display graphical information for a user interface provided via the input/output device 840.

The memory 820 is a computer readable medium such as volatile or non-volatile that stores information within the computing system 800. The memory 820 can store data structures representing configuration object databases, for example. The storage device 830 is capable of providing persistent storage for the computing system 800. The storage device 830 can be a floppy disk device, a hard disk device, an optical disk device, or a tape device, or other suitable persistent storage means. The input/output device 840 provides input/output operations for the computing system 800. In some example embodiments, the input/output device 840 includes a keyboard and/or pointing device. In various implementations, the input/output device 840 includes a display unit for displaying graphical user interfaces.

According to some example embodiments, the input/output device 840 can provide input/output operations for a network device. For example, the input/output device 840 can include Ethernet ports or other networking ports to communicate with one or more wired and/or wireless networks (e.g., a local area network (LAN), a wide area network (WAN), the Internet).

In some example embodiments, the computing system 800 can be used to execute various interactive computer software applications that can be used for organization, analysis and/or storage of data in various formats. Alternatively, the computing system 800 can be used to execute any type of software applications. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities or can be standalone computing products and/or functionalities. Upon activation within the applications, the functionalities can be used to generate the user interface provided via the input/output device 840. The user interface can be generated and presented to a user by the computing system 800 (e.g., on a computer screen monitor, etc.).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and subcombinations of the disclosed features and/or combinations and subcombinations of several further features disclosed above. In addition, the logic flows depicted in the accompanying figures and/or described herein do not necessarily require the particular order shown, or sequential order, to achieve desirable results. Other implementations may be within the scope of the following claims.

1. A system, comprising:
at least one data processor; and
at least one memory storing instructions, which when executed by the at least one data processor, result in operations comprising:
generating, for a conformer of a molecule, a plurality of augmented samples by at least modifying a three-dimensional structure of the conformer such that each augmented sample of the plurality of augmented samples exhibits a different three-dimensional structure than other augmented samples of the plurality of augmented samples;
training a molecular analysis model to generate a plurality of embeddings by at least generating an embedding for each augmented sample in the plurality of augmented samples, where the training of the molecular analysis model includes reducing a difference between the embedding of each augmented sample such that two augmented samples with different three-dimensional structures have similar embeddings, and where the molecular analysis model is further trained to determine, based at least on the plurality of embeddings, a value of a molecular property for the molecule; and
applying the trained molecular analysis model to determine the value of the molecular property for a second different molecule.

2. The system of claim 1, wherein the training of the molecular analysis model includes reducing a loss function quantifying a distance between two or more embeddings of augmented samples generated from a same conformer of the molecule.

3. The system of claim 1, wherein the training of the molecular analysis model excludes training the molecular analysis model to increase a difference between two or more embeddings of augmented samples generated from different conformers of the molecule.

4. The system of claim 1, wherein the training of the molecular analysis model excludes training the molecular analysis model to increase a difference between two or more embeddings of augmented samples generated from conformers of different molecules.

5. The method of claim 1, wherein the training of the molecular analysis model includes reducing a loss function quantifying a difference between the value of the molecular property for the molecule and a ground-truth value of the molecular property for the molecule.

6. The system of claim 1, further comprising:
training the molecular analysis model to generate an additional plurality of embeddings corresponding to an additional plurality of augmented samples associated with an additional conformer of the molecule while minimizing a difference between the additional plurality of embeddings of the additional conformer but not a difference between the plurality of embeddings of the conformer and the additional plurality of embeddings of the additional conformer.

7. The system of claim 1, wherein the molecular analysis model includes a machine learning model trained to generate the embedding for each augmented sample in the plurality of augmented samples, and wherein the molecular analysis model further includes an additional machine learning model trained to determine, based at least on the embedding for each augmented sample, a respective value of the molecular property for each augmented sample.

8. The system of claim 7, wherein the molecular analysis model determines, based at least on the respective value of the molecular property for each augmented sample, the value of the molecular property for the molecule.

9. The system of claim 1, wherein the plurality of augmented samples includes an first augmented sample having a first modification to the three-dimensional structure of the conformer and a second augmented sample having a second modification to the three-dimensional structure of the conformer.

10. The system of claim 9, wherein each of the first modification and the second modification include a change to one or more of an atomic position, a bond angle, a bond length, and a dihedral angle present in the three-dimensional structure of the conformer.

11. The system of claim 10, wherein the change includes adding noise to the one or more of the atomic position, the bond angle, the bond length, and the dihedral angle present in the three-dimensional structure of the conformer.

12. The system of claim 9, wherein the plurality of augmented samples further include a third augmented sample having a third modification to the three-dimensional structure of the conformer.

13. The system of claim 12, wherein the molecular analysis model is further trained to at least
generate an embedding of the third augmented sample while reducing a difference between the embedding of the third augmented sample and each of an embedding of the first augmented sample and an embedding of the second augmented sample, and
determine, based at least on the embedding of the third augmented sample, the value of the molecular property for the molecule.

14. The system of claim 1, wherein the molecular analysis model is trained to perform a classification task or a regression task in order to determine the value of the molecular property.

15. The system of claim 1, wherein the molecular property includes binding affinity, absorption, distribution, metabolism, potency, efficacy, phenotypic effects, or excretion.

16. The system of claim 1, wherein the trained molecular analysis model determines the value of the molecular property of the different molecule by at least
generating, for a conformer of the different molecule, a first augmented sample and a second augmented sample by at least modifying a three-dimensional structure of the conformer of the different molecule;
generating an embedding for the first augmented sample and an embedding for the second augmented sample,
determining, based at least on the embedding of the first augmented sample, the value of the molecular property for the first augmented sample,
determining, based at least on the embedding of the second augmented sample, the value of the molecular property for the second augmented sample,
determining, based at least on the value of the molecular property for each of the first augmented sample and the second augmented sample, the value of the molecular property for the conformer of the additional molecule; and
determining, based at least on the value of the molecular property for the conformer of the additional molecule, the value of the molecular property for the molecule.

17. The system of claim 1, wherein the conformer of the molecule is selected from a conformer ensemble including a plurality of conformers associated with the molecule, and wherein the plurality of conformers have a same chemical composition but differ in structure via one or more rotations around intramolecular bonds.

18. The system of claim 1, further comprising:
training the molecular analysis model based at least on a subset of conformers comprising a random selection of conformers from a conformer ensemble of the molecule.

19. A computer-implemented method, comprising:
generating, for a conformer of a molecule, a plurality of augmented samples by at least modifying a three-dimensional structure of the conformer such that each augmented sample of the plurality of augmented samples exhibits a different three-dimensional structure than other augmented samples of the plurality of augmented samples;
training a molecular analysis model to generate a plurality of embeddings by at least generating an embedding for each augmented sample in the plurality of augmented samples, where the training of the molecular analysis model includes reducing-a difference between the embedding of each augmented sample such that two augmented samples with different three-dimensional structures have similar embeddings, and where the molecular analysis model is further trained to determine, based at least on the plurality of embeddings, a value of a molecular property for the molecule; and
applying the trained molecular analysis model to determine the value of the molecular property for a different molecule.

20. A non-transitory computer readable medium storing instructions, which when executed by at least one data processor, result in operations comprising:
generating, for a conformer of a molecule, a plurality of augmented samples by at least modifying a three-dimensional structure of the conformer such that each augmented sample of the plurality of augmented samples exhibits a different three-dimensional structure than other augmented samples of the plurality of augmented samples;
training a molecular analysis model to generate a plurality of embeddings by at least generating an embedding for each augmented sample in the first plurality of augmented samples, where the training of the molecular analysis model includes reducing-a difference between the embedding of each augmented sample such that two augmented samples with different three-dimensional structures have similar embeddings, and where the molecular analysis model is further trained to determine, based at least on the plurality of embeddings, a value of a molecular property for the molecule; and
applying the trained molecular analysis model to determine the value of the molecular property for a different molecule.
